(19) United States
(12) Patent Application Publication
(10) Pub. No.: US 2002/0110842 A1
(43) Pub. Date: Aug. 15, 2002

(54) Photochemical amplified immunoassay

(76) Inventors: Seymon Bystryak, Toronto (CA); Michael Muehleman, Liverpool, NY (US); Hanoch Slor, Ramat Hasharon (IL)

Correspondence Address: MICHAEL P. WILLIAMS, ESQ, ONE LINCOLN CENTER, SYRACUSE, NY, 13202, US

(21) Appl. No.: 09/784,572

(22) Filed: Feb. 15, 2001

(51) Int. Cl. G01N 33/53; G01N 33/537; G01N 33/543

(52) U.S. Cl. 435/7.92

(57) ABSTRACT

An assay for the determination of an analyte in an aqueous sample includes the steps of binding a first entity having an affinity for the analyte to a solid support. The first entity is bonded with the analyte to form a first complex. The first complex is reacted with a second entity to produce a second complex that is tagged with an enzyme. The second complex is combined with a substrate wherein a third complex is formed. An amplification reagent is added. The sample is irradiated with photonic energy, whereby the combination of the amplification reagent and the photonic energy provides catalysis for the further production of the third complex. The absorbance (OD) of the sample is then measured.

### FIELD OF THE INVENTION

This invention relates to chemical testing and more specifically relates to biochemical assays that test for the presence of selected substances in a sample.

### BACKGROUND OF THE INVENTION

There are known in the art various biochemical methods for the detection of specific substances in samples of blood, serum or tissue. For example, in the field of medical diagnostics there are various tests that can be performed to detect the presence of a foreign substance in blood, serum, saliva, urine or other bodily tissues. More specifically, many of these tests are based on immunologic reactions or antigen- antibody reactions.

The components of the immune system called antibodies are found in the liquid portion of the blood and help protect the body from harm. Antibodies are extremely specific in their reactivity with other chemicals and can be used in a laboratory setting to help diagnose diseases caused by malfunctions of the immune system or the invasion into the body by foreign infections, be they biologic or environmental.

One specific test that is of particular relevance to the present invention is the enzyme-linked immunosorbent assay (ELISA). ELISA is a highly sensitive technique for detecting and measuring antigens or antibodies in a solution. The solution is run over a surface to which immobilized antibodies specific to the target substance have previously been attached, and if the target substance is present it will bind to the antibody layer. Its presence is verified and visualized with an application of secondary antibodies that have been tagged in some way. Currently, ELISA tests are used in the diagnosis of a large number of infections and diseases, most notably HIV, hepatitis and some cancers.

More specifically, ELISA tests typically use a solid phase such as a plastic reaction vessel or plate which is coated with either killed or neutralized virus, or synthetic peptide fragments. Blood samples are then applied to the solid phase and any antibodies to the virus, which suggest viral exposure, will bind to the viral antigen on the solid phase. After a wash step, a second antibody, labeled with an enzyme is added. This antibody is specific and binds to the sample antibody which has already bound to the viral antigen. The presence of the antibody is detected by measuring the amount of the enzyme labeled material, which has specific characteristics such as radioactivity, a specific spectral reflectance, or illuminescence. The most commonly used measurement vehicles pick up the presence of the labeled enzyme by measuring for colorimetry, radioactivity, fluorescence and/or chemilluminescence.

The method of detection which is most relevant to the present invention is colorimetry. Colorimetry is used in a variety of immunoassays, such as competitive assays, sandwich assays, or ELISA assays. Colorimetry is achieved by the attachment of an enzyme to an antigen for the competitive immunoassay methods, attachment to a specific antibody for an immunometric or sandwich assay, and to a generic secondary antibody for an ELISA approach. The most commonly used enzymes are Horseradish Peroxidase (HRP) or Alkaline Phosphatase (AP). HRP is used extensively for detection because it generates large signals from the production of the colored products in the presence of hydrogen peroxide when in use with a large variety of substrates. HRP has high turnover characteristics and good stability, but it is inactivated by a wide range of nucleophiles, such as azide, cyanide, fluoride and hydroxyl ions. HRP also suffers from being susceptible to inactivation from routine buffer constituents. Additionally, the heme group of HRP can form a complex with peroxide which leads to rapid loss of enzymatic activity.

Each of the enzyme systems liberate a soluble or insoluble colored product by incubation with a suitable substrate. The amount of color generated is then measured after a fixed incubation time at a specific wavelength. The optical density (OD) at the specific wavelength is then related back to the concentration of the antigen in the sample.

Although ELISA tests are very sensitive, it is desirable to have tests that are even more sensitive. For instance, it is well known that early detection of infections such as HIV or cancer provides the patient and medical treatment personnel with increased options and opportunities for varied treatments. In addition, early detection of communicable diseases is important in that it can decrease the rate of transmission.

In efforts to reach lower concentrations, or to obtain faster results, fluorometric and chemiluminescent methods of detection have been used. In fluorometric detection, the linked enzyme is a substance that emits light at a certain wavelength (emission wavelength) when it is illuminated by light of a different wavelength (excitation wavelength). Alternatively, in chemiluminescent detection, the linked enzyme is a substance that generates light through a chemical reaction and as such does not use any light source.

Although each of the above-described techniques are sensitive, it is axiomatic that the more sensitive the test, the better. It is well understood by those skilled in the art that if mere detection of a substance is the goal of the assay then other techniques can be utilized, particularly amplification techniques. Some amplification techniques utilize chemical catalysts or in some specialized cases use polymerase chain reaction (PCR), which is a method for amplifying a DNA base sequence. These methods are extremely sensitive, although in some cases these methods are difficult to carry out and very expensive.

Another type of amplification technique utilizes light or photonic energy to increase the sensitivity of the assay. This technique is disclosed in U.S. Pat. No. 5,776,703 to Bystryak. It is widely known that some chemical reactions are photosensitive dependent upon the quantum chemical structure and other properties of the reactants. The rate of a photosensitive reaction is much higher if the reaction mixture is illuminated by an intense light of a specific wavelength compared to a similar reaction taking place in the absence of such light. Consequently, if a reaction taking place in a diagnostic assay can be made photosensitive, the sensitivity of such assay can be substantially enhanced.

The technique of Bystryak includes the binding of an antibody to a suspected antigen wherein the antibody is labeled with an enzyme such as HRP and added to a biological liquid, for example blood or serum. A portion of the HRP-labeled antibodies binds with the antibodies that are specific to the antigen and existing already in the biological liquid to form an [antibody]-[antigen]-[HRP labeled antibody] complex. Subsequently, after an incubation time, the HRP-labeled antibody which did not bind to the Ab-Ag complex is removed from the solution by rinsing or washing. Then, a substrate solution, for instance, H₂O₂ and o-PD (orthophenylenediamine), is added to the test tube and the o-PD is oxidized with HRP acting as an oxidizing catalyst. The oxidation product in the event of HRP and o-PD is diaminophenazine (DAP). DAP is a colored substance, the optical density of which can be read with the aid of a photometer. At this point in the assay a stopping solution, such as sulfuric acid, is used in order to stop the various chemical reactions from proceeding and producing reaction products that may interfere with an accurate measurement. The result is proportional to HRP bound to antibodies and therefore to the added antibodies forming the Ab-Ag-Ab complex.

Bystryak further discloses that the procedure performed to this point can be enhanced by the application of intense light at the wavelength of 400 to 500 nm. Prior to spectrophotometer reading, the test tube is illuminated by an intense source of light of a wavelength in the above range. The DAP obtained in the first stage of the reaction together with the light photons serve as new catalyzing agents for further production of DAP. Thus a two stage reaction takes place:

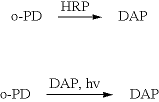

The resulting optical density of the sample is measured by a spectrophotometer and the results obtained by Bystryak are an improvement over the prior art. However, when utilizing the method of Bystryak, if the reaction is stopped with sulfuric acid as described above the results are not satisfactory, probably because the conversion to DAP is restricted. And conversely, if the Bystryak method reaction is not stopped at this point the background noise created by the secondary chemical reactions in the solution severely restricts the utility of the method.

Although the method of Bystryak increased the sensitivities of assays versus prior art methods, it is still desirable to have an even more sensitive technique. In addition to sensitivity, it would be beneficial to have an immunoassay method that also improved the signal-to-noise (S/N) ratio, which would substantially increase the effective range of the known methods. The method of Bystryak is adversely affected to a large degree by background noise. In addition, the method of Bystryak is highly dependent upon a number of factors including: (1) the wavelength of the light used to catalyze the reaction; (2) the intensity of the light (the number of photons used to catalyze the reaction); and, (3) the illumination exposure (intensity X time).

It is therefore desirable to have a method of detecting an analyte in a sample wherein the method has increased sensitivity compared to known methods. It is also desirable to have a method of detecting an analyte in a sample with an improved S/N ratio compared to known methods. It is also desirable to have a method for detecting an analyte in a sample that can be used with existing ELISA methodology.

### OBJECTS AND SUMMARY OF THE INVENTION

It is therefore an object of the invention to provide a method of detecting an analyte in a sample wherein the method has increased sensitivity compared to known methods.

It is another object of the invention to provide a method for detecting an analyte in a sample with an improved S/N ratio compared to known methods.

It is yet another object of the invention to provide an improved method for detecting an analyte in a sample wherein the method is compatible with existing ELISA methodology.

These and other objects are obtained by providing a method for performing an assay for the determination of an analyte in an aqueous sample including the steps of binding a first entity having an affinity for the analyte to a solid support. The first entity is bonded with the analyte to form a first complex. The first complex is reacted with a second entity to produce a second complex that is tagged with an enzyme. The second complex is combined with a substrate wherein a third complex is formed. An amplification reagent is added. The sample is irradiated with photonic energy, whereby the amplification reagent and the photonic energy provide catalysis for the further production of the third complex. The absorbance (OD) of the sample is now measured.

### DETAILED DESCRIPTION

A detailed description of the invention is presented herein. The method is further described as it applies to a commercially available ELISA test which is meant only to be illustrative of the method and not to limit the scope of the claims that follow. One skilled in the art would recognize the utility of the present invention in other similar assays.

The methodology of the test closely follows the protocols of commercially available immunoassay tests, for example, the DuPont HIV p24 ELISA test currently supplied by NEN Life Science Products, Boston, Mass. It is well understood by those skilled in the art that the present invention will have numerous applications in the field of immunoassay testing and the scope of the invention will become more apparent through the following example.

As an initial step, at least one interactive material, typically an antibody, is retained or bound to a support member which may comprise a plate, a body of beads or other particulate matter, tubes, rings, a porous matrix such as those used in Western Blot techniques, or other materials of various design known to those skilled in the art. In the case of the Dupont p24 ELISA kit, the plate is a microplate well specifically designed for the assay by coating the well with a highly specific mouse monoclonal antibody.

Next, a sample of the biological fluid that is being tested is added to the plate. Typically the sample is blood, serum or a cell culture. If the biological fluid contains the target analyte (antigen) then an antibody-antigen complex is formed. In the Dupont p24 ELISA kit, the serum/plasma sample may or may not require immune complex disruption.

Next, an interactive material such as an antibody to the specific antigen is added to the plate. This second antibody has been labeled and as discussed above, it is well known in the art to use various enzymes as labels. Of particular relevance to the present invention, the enzyme used becomes part of a complex that reacts with a substrate to produce a third complex which is detectable under colorimetric study. As an example, the Dupont p24 kit uses a streptavidin protein labeled with HRP which reacts with o-PD to form DAP, which produces a yellow color that is detectable by calorimetric or absorbance photometers.

In the prior art methods, a stopping agent would now be applied after a suitable reaction time. For example, in the Dupont p24 kit, the stopping agent is sulfuric acid H₂SO₄. In contrast, applicant has discovered that removing the step of adding the stopping agent and instead using an amplification reagent in combination with photonic energy at this point in the prior art method greatly amplifies the production of the third complex, and thereby increases the sensitivity of the test. The present invention varies from the standard protocol and other prior art methods by providing the amplification reagent and by eliminating the stopping step. For example, when using the Dupont p24 kit with the method of the present invention, the reaction is not stopped with sulfuric acid and instead an amplification reagent is added and coupled with a dose of photonic energy. This serves to catalyze the production of DAP and greatly increases the sensitivity of the assay.

To date, the Applicants best mode of practicing the invention includes as an amplification reagent the commercially available detergent Triton X-100 at a concentration in the range of 10% in a phosphate/citrate buffer with a pH of approximately 5.0. Triton X-100 has a chemical formula of C₁₄H₂₂O (C₂H₄O)n where the average number of ethylene oxide units per molecule ranges from 9 to 10.

Applicants have also used Tween, another commercially available detergent, as an amplification reagent with results that are improved over the prior art but to date are not as successful as Triton. To date, it is not well understood which particular characteristics of the amplification reagent contribute to the success of the present invention but it is known that the concentration and pH of the above identified amplification reagents has significant impacts on the outcome of the assay. However, the present invention is not limited to any one amplification reagent, but the method can utilize as yet undetermined amplification reagents that when used in combination with photonic energy of various wavelengths will act as a catalyst in the production of a complex that is detectable using any of the known detection techniques.

Applicants have also discovered that the dose of photonic energy that is applied during the assay method can be either a constant dose or a pulsed dose with varying results. The dose of photonic energy is dependent upon the intensity of the photons and the time of exposure, and therefore the dose can be quantified by photons per second. The photons are supplied by light panels, top and bottom, that are positioned above and below the plate. The panels are six inches by six inches which allows for a standard plate to be fully illuminated. The dose is defined as the radiance of the panels times the illumination duration is seconds. In the preferred embodiment, the intensity of each panel corresponds to 19,000 candela per square meter, which is herein referred to as 100% intensity.

In the preferred embodiment, the photonic energy is applied at a wavelength in the range of 450 to 500nm, or portions thereof, wherein the assay is exposed to a dose of 50% intensity for a period of time of sixty (60). The assay is then not exposed to any photonic energy for a period of time of ten (10) seconds. The time of exposure coupled with the time of non-exposure constitutes one cycle and the preferred embodiment utilizes a series of approximately ten (10) cycles, or so-called pulses.

The preferred detection technique is colorimetric detection using a photometer. For example, using the Dupont HIV p24 kit as an example, the absorbance or optical density (OD) of the sample is measured at 450 nm which indicates the concentration of DAP, and therefore the presence of HIV antibodies in the blood sample. Of course, one skilled in the art would recognize that the OD is measured at a wavelength that detects the presence of the third complex formed in any particular assay method.

The increased production of DAP in the example is achieved without the production of limiting background noise. Therefore, the utility of the method is greatly increased over the prior art.

In accordance with another aspect of the invention, there is provided a reagent kit or package of materials for accomplishing an assay for an analyte in accordance with the method of the invention. The kit may include a solid support having attached thereto an antianalyte specific to the analyte. The kit may also include standards for the analyte, as, for example, one or more analyte samples of known concentration, or it may include other reagents or solutions, such as buffers, or labeled specific antigens, antibodies or complexes thereof useful in carrying out the assay. The kit may also contain an amplification reagent. The components of the kit may be supplied in separate containers, as, for example, vials, or two or more of the components may be combined in a single contained

We claim:

1. A method for performing an assay for the determination of an analyte in a sample, the method comprising:
providing a first entity having an affinity for the analyte;
bonding said first entity with the analyte to form a first complex;
reacting said first complex with a second entity to produce a second complex, said second entity having a tag;
contacting said second complex with a substrate whereby a third complex is formed;
adding an amplification reagent;
irradiating the sample with photonic energy whereby said amplification reagent and said photonic energy provide catalysis for the further production of said third complex; and, measuring the sample.

2. The method of claim 1 wherein said first entity comprises a primary antibody.

3. The method of claim 2 wherein said primary antibody is bound to a support.

4. The method of claim 1 wherein said second entity comprises a secondary antibody.

5. The method of claim 4 wherein said tag comprises an enzyme.

6. The method of claim 1 wherein said amplification reagent comprises a detergent.

7. The method of claim 6 wherein said detergent comprises Triton X-100 at a concentration of 10% in a phosphate/citrate buffer having a pH of approximately 5.0.

8. The method of claim 1 wherein said photonic energy is applied in a pulsed dose.

9. The method of claim 1 wherein said measuring step comprises measurement of absorbance (OD) using a photometer.

10. A method for performing an enzyme-linked immunosorbent assay for the determination of an antigen in an aqueous sample, the method comprising:
binding a primary antibody to a plate;
binding the antigen to the primary antibody to form a first complex;
reacting the first complex with a secondary antibody entity to produce a second complex, said second entity tagged with horseradish peroxidase;
contacting the second complex with oPD (orthophenylene-diamine) and H202 by which oPD is converted to DAP (2,3-diamino-phenazine);
adding an amplification reagent, said amplification reagent including Triton X-100 at a concentration of 10% in a phosphate/citrate buffer having a pH of approximately 5.0;
irradiating the sample with a pulsed dose of photonic energy in the range of 400-500nm, whereby the combination of said amplification reagent and said photonic energy provides catalysis for the further production of DAP; and,
measuring the absorbance (OD) of the sample at 450nm.

11. A kit for performing an enzyme-linked immunosorbent assay including the step of irradiating the sample with a pulsed dose of photonic energy, the assay used for the determination of an antigen in an aqueous sample, the kit comprising:
a primary antibody bound to a plate;
an antigen to said primary antibody;
a secondary antibody tagged with an enzyme; and, an amplification reagent.

12. The kit of claim 11 wherein said amplification reagent comprises a detergent.

13. The kit of claim 12 wherein said detergent comprises Triton X-1 00 at a concentration of 10% in a phosphate/citrate buffer having a pH of approximately 5.0.

14. The kit of claim 11 wherein said enzyme comprises Horseradish Peroxidase.

15. The kit of claim 14 wherein said substrate comprises oPD (orthophenylenediamine) and H2O2.

16. A kit for performing an enzyme-linked immunosorbent assay including the step of irradiating the sample with a pulsed dose of photonic energy, the assay used for the determination of an antigen in an aqueous sample, the kit comprising:
a primary antibody bound to a plate;
an antigen to said primary antibody;
a secondary antibody tagged with Horseradish Peroxidase;
a substrate comprising oPD (orthophenylene-diamine) and H2O2 an amplification reagent comprising Triton X-100 at a concentration of 10% in a phosphate/citrate buffer having a pH of approximately 5.0.
